(19) United States
(12) Patent Application Publication
(10) Pub. No.: US 2004/0028738 A1
(43) Pub. Date: Feb. 12, 2004

(54) Process for the preparation of porous collagen matrix

(75) Inventors: Lynn L. H. Huang, Tainan (TW); Hsyue Jen Hsieh, Taipei (TW); Po Yang Chen, Tainan (TW)

Correspondence Address: LADAS & PARRY, 26 WEST 61ST STREET, NEW YORK, NY, 10023, US

(73) Assignee: LYNN L.H. HUANG

(21) Appl. No.: 10/426,891

(22) Filed: Apr. 30, 2003

Related U.S. Application Data
(63) Continuation-in-part of application No. 09/723,696, filed on Nov. 28, 2000, now abandoned.

(30) Foreign Application Priority Data
Oct. 5, 2000 (TW) 089120767

(51) Int. Cl. A61K 9/14

(52) U.S. Cl. 424/484

(57) ABSTRACT

The subject invention discloses a process for the preparation of a porous collagen matrix. Said process comprises providing a neutral or nearly neutral collagen solution, incubating the collagen solution at a temperature of between about 30 and about 45° C. for a period of time sufficient to reconstitute collagen fibrils to obtain a collagen gel matrix, freezing said collagen gel matrix with an appropriate temperature reduction rate to an appropriate freezing temperature, lyophilizing said matrix to form a porous collagen matrix and treating the lyophilized collagen matrix with an organic solvent that can quickly penetrate into the collagen matrix to prevent the shrinkage thereof.

The invention also provides a process for the preparation of a porous collagen matrix with different pore sizes by selecting the collagen solution or controlling freezing temperature or temperature reduction rate.

The invention also provides a porous collagen matrix prepared by said processes.

The subject application is a continuation application of U.S. Ser. No. 09/723,696 filed on Nov. 28, 2000.

### FIELD OF THE INVENTION

Collagen is a biodegradable protein and exists in the form of fibers in connective tissues of most animals. The primary function of collagen is to maintain the integrity of tissues and to provide tension essential to tissues. Collagen molecule is a biological macromolecule composed of three polypeptide chains that twist around one another. Each polypeptide is composed of about one thousand amino acids, wherein the primary amino acids are glycine, proline and hydroxyproline. At present, at least 21 different types of collagen are found.

For applications, collagen can be manufactured to different forms, such as sponge, gel, tube, sheet, etc. They can be applied in wound dressings, drug carriers, scaffold of artificial organs, microcarriers and macrocarriers for supporting cell growth, and for hemostasis and recovery of tissues, etc. In order to make the above collagen matrix exhibiting a porous structure to facilitate cell migration, cell growth or encapsulation and release of drugs, the pores of the matrix are normally formed through a lyophilization step. Generally, during the lyophilization step, the materials are frozen at a temperature of −80° C. or rapidly frozen by liquid nitrogen and then subject to vacuum dry. Because ice crystals produced during rapid freezing are smaller, the pore size of the matrix obtained is usually small (less than 30 μm).

The preparation of porous collagen matrix has been disclosed in many prior art patents. For instance, in U.S. Pat. No. 4,193,813, comminuted collagen at pH 3.5 to pH 6.5 is crosslinked with glutaraldehyde followed by freezing at 0 to −20° C. After thawing, the water of the frozen material is eliminated to form a sponge matrix. The pore size of the matrix formed by this process is about 80˜1400 μm. U.S. Pat. No. 4,412,947 relates to a process that pure insoluble particulate collagen is suspended in a weak aqueous organic acid solution followed by freezing at −60 to −70° C. with a temperature reduction rate of −0.3 to −0.4° C. per minute, and then lyophilized to form a porous collagen sheet. U.S. Pat. No. 4,522,753 relates to a process of mixing collagen and chrondroitin sulfate to form a copolymer material. The material is then cross-linked by glutaraldehyde and lyophilized to form a porous matrix with a pore size of 20˜180 μm. Such matrix can be used as a basic material of synthetic skin. U.S. Pat. No. 4,970,298 discloses a collagen matrix prepared by dispersing collagen in an acidic solution or by mixing the collagen dispersion with hyaluronic acid and fibronectin. The dispersion is frozen at the different temperatures and then lyophilized to form a porous sponge. The sponge is cross-linked with a carbodiimide or by a dehydrothermal process. The freezing temperature is −30° C. to −50° C. The pore size of the matrix obtained is about 20˜250 μm. The collagen matrix containing hyaluronic acid or fibronectin exhibits a pore size of 100˜150 μm. U.S. Pat. No. 4,948,540 describes a process that involves freeze-drying the mixture of native collagen and soluble collagen fibers and compressing at a pressure of 15,000˜30,000 p.s.i. The material is then cross-linked by a dehydrothermal method to obtain a final product which is a sheet material with high absorptivity. U.S. Pat. No. 5,116,552 describes a process for preparing a crack-free sponge matrix. An acidic collagen solution is frozen at −40° C. and lyophilized into a sponge. The sponge is then incubated at 105° C. for 24 hours and then cross-linked for 24 hours with glutaraldehyde to form a matrix with a pore size of 50˜120 μm. The matrix is then immersed in 15% alcohol. After the second lyophilization at a lower temperature of −80° C. or −135° C., a crack-free sponge matrix is obtained. U.S. Pat. No. 5,869,080 describes a process for preparing an absorbable implant material. A sponge matrix is formed by adding a proper amount of alcohol to the collagen dispersion in sodium hydroxide, pre-freezing it at a low temperature (about −5° C.), adding ice particles to the dispersion, cross-linking the dispersion with hexamethylene diisocyanate (HMDI), and followed by lyophilizing the dispersion. The matrix obtained by this process exhibits a pore size of 50˜400 μm.

Chemical cross-linking agents are generally utilized in the preparation of a collagen matrix so as to reinforce the structural strength of such collagen matrix. However, most cross-linking agents contain highly reactive functional groups. If the cross-linking agents are not completely reacted with the collagen matrix, the residual cross-linking agents may induce in vivo cytotoxicity when the collagen matrix is applied in human body. For instance, U.S. Pat. No. 4,233,360 discloses the use of formaldehyde as a cross-linking agent to treat collagen materials, but no elimination of residual or non-reacted formaldehyde is mentioned. Speer, D. P. et al. describes the use of glutaraldehyde in cross-linking collagen materials. It is observed that the solubility, antigenicity and biodegradation of the collagen matrix are effectively reduced by a glutaraldehyde treatment. Furthermore, it is also observed that fibroblast growth in tissue culture is inhibited and foreign body giant cell reaction to bioimplants of the collagen matrix is induced. Koob, T. J. et al. describes the use of nordihydroguaiaretic acid (NDGA) in cross-linking collagen matrix. To reduce the cytotoxicity of NDGA to fibroblasts, the cross-linked collagen matrix must be repeatedly washed by use of 70% alcohol and PBS solution, and the concentration of the NDGA used should be less than 100 μM. Gough, J. E. et al. describes the use of glutaraldehyde in cross-linking collagen/poly(vinyl alcohol) to make bioartificial composite films. It is found that human osteoblasts undergo apoptosis on glutaraldehyde crosslinked films, and higher collagen content results in a higher level of apoptosis with poor cell attachment and spreading of remaining cells. Given the above, it is known that the use of a cross-linking agent in the preparation of collagen matrix may induce cytotoxicity to human body.

U.S. Pat. Nos. 5,110,604 and 5,024,841 disclose a collagen matrix and methods for preparation of such collagen matrix without the use of any chemical cross-linking agents. The collagen matrix is prepared by providing an acidic aqueous solution of atelopeptide collagen, precipitating the collagen from the solution by raising the pH of the solution to form a dispersion of the precipitated collagen fibrils, casting the dispersion and then flash-freezing and lyophilizing the case dispersion. Instead of chemical cross-linking agents, said process utilizes dehydrothermal treatment at a temperature of 60 to 120° C. to enhance the structural strength of the collagen matrix. However, Kevin S. W. et al. describes that dehydrothermal treatment may cause fragmentation of at least one portion of the collagen molecules. Thus, the use of dehydrothermal treatment in the preparation of collagen matrix still has some disadvantages. Furthermore, in the processes of the two US patents, the pH value of collagen-containing solution must be raised so as to precipitate the collagen molecules, and then obtain a homogenous dispersion with a high concentration of precipitated collagen fibrils. A high concentration of collagen fibrils may thus reinforce the structural strength of the collagen matrix. Thereafter, a sheet of collagen matrix with a desired thickness is formed by casting and subject to the following lyophilization. The processes for preparing the collagen matrix without using a chemical cross-linking agent disclosed in the two US patents are quite complicated and laborious.

In addition, U.S. Pat. No. 5,514,181 discloses the addition of alcohol in the lyophilization of collagen matrix as an anti-freeze substance to obtain a collagen matrix with smaller pores. The purpose of adding alcohol in the lyophilization of collagen matrix is the same as that in U.S. Pat. Nos. 5,869,080 and 5,116,552. However, Dagalakis N. et al. mentions that the drying of collagen matrix in an alcohol solution may cause shrinkage of said collagen matrix, similar to what we observed in alcohol added matrix prior to lyophilization. The alcohol added prior to lyophilization can not exceed 20% for −20° C. freezing and 50% for −80° C. freezing in order to obtain a solid matrix with ice crystals for subsequent lyophilization.

There is a need for the market to develop a process for easily preparing collagen matrix without using any chemical cross-linking agents.

### SUMMARY OF THE INVENTION

In order to improve the disadvantages of conventional techniques for preparing a collagen matrix, to obtain a better pore homogeneity, and to prevent possible toxic effects resulted from the chemical cross-linking agents, the subject invention proposes an improved process for manufacturing the porous collagen matrix, which can be processed into collagen-related products. The subject invention utilizes uncross-linked neutral or close to neutral collagen solution as the materials. The collagen solution is simply reconstituted in a desired container at a temperature relatively low to dehydrothermal treatment to form a collagen gel matrix, without additional precipitation of collagen fibrils used in the prior art and without the use of cross-linking agents. Thereafter, the collagen gel matrix is lyophilized and treated with an organic solvent, such as absolute ethanol or acetone. Thus, a porous collagen matrix with good pore homogeneity and stability and desired pore sizes can be easily prepared.

Furthermore, according to the invention, the pore size of the collagen matrix can be controlled by selecting or controlling the different operation parameters in the preparation process, such as by selecting the species of buffer or salt content in the collagen solution, or controlling the freezing temperature or temperature reduction rate in the freezing step.

An object of the subject invention is to provide a process for preparation of a porous collagen matrix.

Another object of the invention is to provide a process for preparation of a porous collagen matrix with a desired pore size.

Still another object of the invention is to provide a porous collagen matrix prepared by the methods of the invention.

Additional objects and features of the present invention will become more apparent and the invention itself will be best understood from the following Detailed Description of the Invention, when read with reference to the accompanying drawings.

### BRIEF DESCRIPTION OF THE DRAWINGS

FIG. 1 shows porous collagen matrices prepared by the process that reconstituted collagen matrices were formed from neutralized acetic acid solution, frozen with a rapid temperature reduction rate and lyophilized, wherein (A) shows the matrix frozen to −20° C.; (B) shows the matrix frozen to −40° C.; and (C) shows the matrix frozen to −80° C.

FIG. 2 shows porous collagen matrices prepared by the process that reconstituted collagen matrices were formed from neutralized acetic acid solution, frozen with a slow temperature reduction rate and lyophilized, wherein (A) shows the matrix frozen to −20° C.; and (B) shows the matrix frozen to −80° C.

FIG. 3 shows scanning electron micrographs of a porous collagen matrix prepared by the process that a reconstituted collagen matrix was formed from neutral phosphate buffered saline solution with 0.135 M NaCl (PBS), frozen to −20° C. with a rapid temperature reduction rate and lyophilized, wherein (A) is a photograph with magnification of 50; and (B) is a photograph with magnification of 400.

FIG. 4 shows porous collagen matrices prepared by the process that reconstituted collagen matrices were formed from neutral phosphate buffered saline solution, frozen to −20° C. with a rapid temperature reduction rate and lyophilized, wherein (A) shows that the solution comprised 0.5M sodium chloride; and (B) shows that the solution comprised 1.0M sodium chloride.

FIG. 5 shows the influence of solvents on the pore structure on the surface of the matrix, wherein (A) shows that the matrix was immersed in glutaraldehyde solution; and (B) shows that the matrix was immersed in absolute alcohol.

### DETAILED DESCRIPTION OF THE INVENTION

The below description providing various embodiments and specific details of the invention is illustrative only and should not be construed in any way as limiting the invention. Furthermore, various applications of the invention, and modifications thereto, which may occur to those who are skilled in the art, are also encompassed by the general concepts described below.

It is an aspect of the invention to provide a process for the preparation of a porous collagen matrix, which comprises providing a neutral or nearly neutral collagen solution, incubating the collagen solution at a temperature between about 30 and about 45° C. for a period of time sufficient to reconstitute collagen fibrils to obtain a collagen gel matrix, freezing said collagen gel matrix with an appropriate temperature reduction rate to an appropriate freezing temperature, lyophilizing said matrix to form a porous collagen matrix and treating the lyophilized collagen matrix with an organic solvent that can quickly penetrate into the collagen matrix to prevent the shrinkage thereof.

Collagen is rich in the connective tissues of animals, such as cow, goat, pig or chicken. Collagen materials can be extracted and purified from the connective tissues of animals by any process known in the art, or obtained through biotechnology, such as the genetically recombinant collagen. According to one preferred embodiment of the invention, the collagen of porcine hide can be extracted and purified by homogenizing the shreds of de-furred and de-lipid porcine hide and dissolved in an acid solution, removing the end-terminal antigen of the collagen molecule by pepsin, and adjusting the solution to neutral to inactivate pepsin, repeatedly salting out, dissolving and dialyzing in an acid or neutral buffer containing 1M of sodium chloride. Thus, a collagen solution is obtained. The acidic solution includes, but is not limited to, acetic acid, citric acid, oxalic acid, hydrogen chloride or sulfuric acid, and preferably acetic acid. In a preferred embodiment of the invention, the acid solution is 0.5M of acetic acid solution.

According to the invention, the collagen solution obtained is prepared to provide a “neutral or nearly neutral” collagen solution. The “neutral or nearly neutral collagen solution” refers to a collagen solution having a pH value ranging from about 5 to about 9. The neutral or nearly neutral collagen solution can be obtained by any process known to persons skilled in the art. For instance, a neutral collagen solution can be prepared by adjusting the pH of the collagen solution to neutral by adding alkaline solution, such as sodium hydroxide solution. Collagen can be also dialyzed to a neutral or nearly neutral buffer in which may contain a certain salt. The buffer includes, but is not limited to, phosphate buffered saline solution, phosphate buffer, sodium or potassium acetate buffer, sodium or potassium citrate buffer, succinate buffer, sodium or potassium bicarbonate buffer, tris(hydroxymethyl)aminomethane buffer, universal buffer (Britton and Robinson type), citrate-phosphate buffer, maleate buffer, imidazole (glyoxaline) buffer, β,β′-dimethylglutarate buffer, 2,4,6-trimethylpyridine (2,4,6-collidine) buffer, triethanolamine buffer, 5,5-diethylbarbiturate buffer, dimethylleucylglycine buffer, or halide buffer (such as sodium chloride), and preferably phosphate buffered saline solution.

The process for reconstitution of collagen in the invention comprises incubating the neutral or nearly neutral collagen solution at about 30 to about 45° C. in an incubator, preferably at about 37° C., for a period of time more than 0.5 hour, preferably about 24 hours, to reconstitute collagen molecules into fibrils and form a collagen gel matrix. The term “collagen gel matrix” refers to a collagen matrix with a gel-like character obtained from a neutral or nearly neutral collagen solution without removal of water upon the reconstitution of collagen fibrils.

The collagen gel matrix is then frozen with a proper temperature reduction rate to an appropriate freezing temperature and then lyophilized to obtain a porous collagen matrix. The term “appropriate temperature reduction rate” refers to a rate of reducing the temperature to obtain a porous collagen matrix with good pore homogeneity and stability and desired pore sizes. For instance, the “temperature reduction rate” refers to a rapid temperature reduction rate, such as the rate more than about −5° C. per minute or a slow temperature reduction rate, such as the rate ranging from about −10° C. to about −60° C. per hour. The term “appropriate freezing temperature” refers to a temperature ranging from about 0 to about −80° C. For instance, the collagen gel matrix of the subject invention is frozen at a temperature of −65° C. and then lyophilized in accordance with the process described in U.S. Pat. No. 4,412,947.

The porous collagen matrix with desired pore sizes can be obtained by means of selecting the components of the neutral or nearly neutral collagen solution and/or controlling the freezing temperature and the temperature reduction rate of the freezing step. The pore size of the porous collagen matrix prepared by the process of the invention depends on the requirement of medical use. Generally, a collagen matrix with pore sizes of 50˜200 μm can be used as the artificial skin, wound dressings, or scaffolds in tissue engineering.

While selecting the components of the neutral collagen solution, the neutral or nearly neutral collagen solution can be further dialyzed into a neutral salt buffer, or be incorporated with a metal salt into said buffer upon reconstitution of the collagen fibrils. The metal salts suitable for the invention include, but are not limited to, sodium chloride, potassium chloride, calcium chloride, ammonium sulfate, magnesium chloride, sodium fluoride, alkaline halide, alkaline earths halide, sodium acetate and sodium carbonate. For instance, if the starting materials are dissolved in a neutral phosphate buffer with a concentration of 0.13M sodium chloride, the average pore size of the porous collagen matrix finally obtained can be more than 100 μm.

The temperature reduction rate and the freezing temperature are both correlated with the pore size and the pore homogeneity of the collagen matrix. With regard to the relationship between the freezing temperature and the pore size of the collagen matrix, for the reconstituted collagen that is in neutralized acetic acid solution, the average pore size of the matrix is larger and is more than about 50 μm when collagen gel matrix is frozen to about −20° C.; the average pore size of the matrix is about 30 μm when the collagen gel matrix is frozen to about −40° C.; and the average pore size of the matrix is more than about 15 μm when the collagen gel matrix is frozen to about −80° C. When the freezing temperature is about −20° C. and about −40° C., the matrix product shows fusion of fibrils; when the freezing temperature is about −80° C., the matrix product exhibits obvious fibrillar structure. When the collagen gel matrix is frozen with a slow temperature reduction rate, the matrix exhibits a range of pore sizes with poor pore homogeneity. The matrix frozen to about −20° C. with a slow temperature reduction rate exhibits a pore size of about 50 to about 300 μm. The matrix frozen to about −80° C. with a slow temperature reduction rate exhibits a pore size of about 15 to about 150 μm. When the collagen gel matrix is frozen with a rapid temperature reduction rate, the matrix exhibits a narrower range of pore sizes with better pore homogeneity. For instance, if the temperature reduction rate is quicker than about −10° C. per minute, the range of the pore size can be controlled within about 30 μm.

In addition, for the reconstituted collagen that is in a neutral PBS solution, the pore size of the matrix is about 100 to about 125 μm when the collagen gel matrix is frozen to about −20° C. with a rapid temperature reduction rate; and the pore size of the matrix is about 30 to about 50 μm when the collagen gel matrix is frozen to about −80° C. When the concentration of sodium chloride in the collagen solution which is dissolved in the neutral phosphate buffer is increased from about 0.135 to about 0.5˜1.0M, the pore size of the matrix is about 30 to about 40 μm when the collagen gel matrix is frozen to about −20° C.

In order to maintain the pore size and structure of the collagen matrix for applications, the process of the invention further comprises treating the lyophilized porous collagen matrix with an organic solvent. The “organic solvent,” which can quickly penetrate into the collagen matrix to prevent the shrinkage thereof, refers to a solvent, which has a smaller surface tension on the collagen fibrils or molecules, and thus can quickly penetrate into the collagen matrix to support the pores of the collagen matrix. When the organic solvent contacts the collagen matrix, the contact angles are almost zero or cannot be detectable, if the surface tension between the solvent and the collagen fibrils or molecules is quite small. The organic solvents suitable for the invention include, but are not limited to, alcohols, such as methanol, ethanol, propanol, isopropanol, butanol or isobutanol; ketones, such as acetone, 2-butanone, cyclohexanone or acetophenone; chloroform, N,N-dimethylformamide or dimethyl sulfoxide. The organic solvent is preferably ethanol and more preferably absolute ethanol, which can completely maintain the pore structure of the collagen matrix.

The subject invention also provides porous collagen matrices prepared by the above-mentioned processes. According to the variation of the above different solutions, temperature reduction rates of freezing, freezing temperatures and concentrations of metal salts, the pore size of the matrix can be controlled. The results are presented in Table 1.

TABLE 1
The pore distribution and pore homogeneity of the porous collagen
matrices obtained by different preparation processes

| | | Temperature | The pore size | |
|---|---|---|---|---|
| | | reduction | of the | |
| | Freezing | rate | collagen | Pore |
| Solution | temperature | of freezing | matrix (μm) | homogeneity |
| Acetic acid | −20° C. | Slow | 50˜300 | Poor |
| solution | | | | |
| Neutralized | −80° C. | Slow | 15˜150 | Poor |
| acetic acid | | | | |
| solution | | | | |
| Neutralized | −20° C. | Rapid | 50˜75 | Good |
| acetic acid | | | | |
| solution | | | | |
| Neutralized | −40° C. | Rapid | 30˜50 | Good |
| acetic acid | | | | |
| solution | | | | |
| Neutralized | −80° C. | Rapid | 15˜40 | Good |
| acetic acid | | | | |
| solution | | | | |
| Neutralized | −20° C. | Rapid | 100˜300 | Very poor |
| acetic acid | | | | |
| solution | | | | |
| (unrecon- | | | | |
| stituted | | | | |
| collagen) | | | | |
| Neutral PBS | −20° C. | Rapid | 100˜125 | Good |
| solution | | | | |
| Neutral PBS | −80° C. | Rapid | 30˜50 | Good |
| solution | | | | |
| Neutral PB | −20° C. | Rapid | 30˜40 | Good |
| solution/ | | | | |
| 0.5 M NaCl | | | | |
| Neutral PB | −20° C. | Rapid | 30˜40 | Good |
| solution/ | | | | |
| 1.0 M NaCl | | | | |

The following examples are for further illustration of the invention but not intended to limit the invention. Any modifications and applications by persons skilled in the art in accordance with the teachings of the invention should be within the scope of the invention.

### EXAMPLE

### Example 1

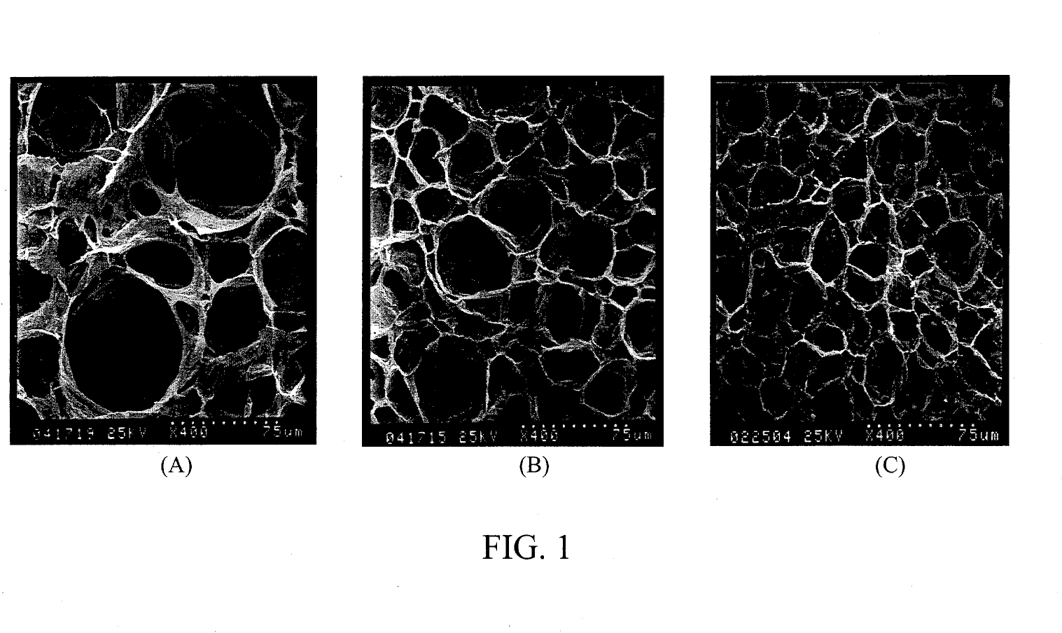

Collagen in 0.5M acetic acid solution was adjusted to pH 7.2 by using sodium hydroxide solution. The solution was placed in a glass container and incubated in a 37° C. incubator for 24 hours to form a reconstituted collagen gel matrix. The collagen gel matrix was frozen with a rapid temperature reduction rate (the temperature reduction rate is 20° C. per minute) to a temperature of −20° C., −40° C. or −80° C. and then lyophilized under vacuum of 10⁻³ to 10⁻⁵ torr until the matrix was dried. The porous collagen matrices with high pore homogeneity were obtained and illustrated in FIG. 1. The pore sizes of these matrices were 50˜75, 30˜50 and 15˜40 μm, respectively.

### Example 2

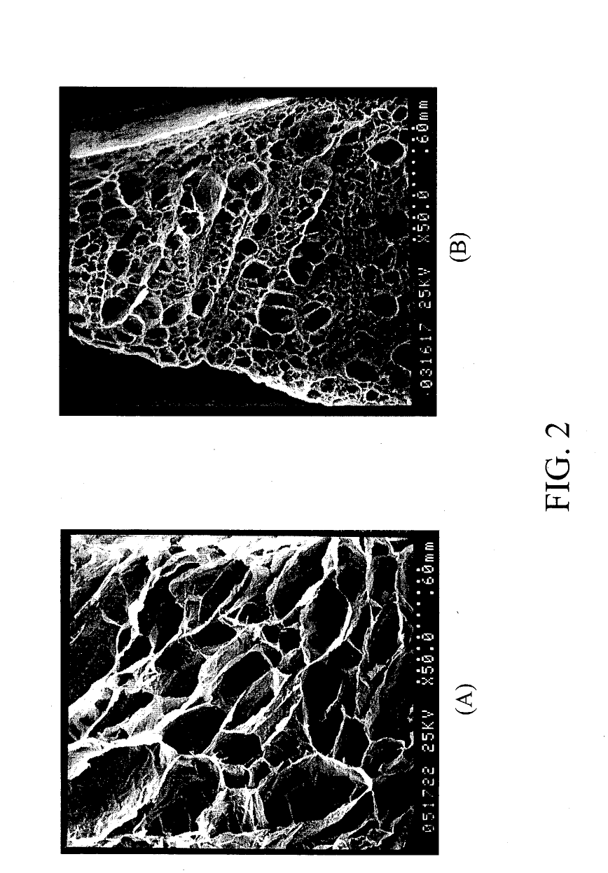

Collagen after purification was dialyzed to a 0.5M acetic acid solution to form an acidic collagen solution. Sodium hydroxide was used to adjust the collagen solution to neutral (pH=7.2). The solution was placed in a glass container and incubated in a 37° C. incubator for 24 hours to form a reconstituted collagen gel matrix. The collagen gel matrix was frozen with a slow temperature reduction rate (the temperature reduction rate is 20° C. per hour) to a temperature of −20° C. to −80° C. and then lyophilized under vacuum of 10⁻³ to 10⁻⁵ torr until the matrices were dried. The porous collagen matrices were obtained which exhibit an average pore size of more than 50 μm. The range of the pore size was significantly large, which meant that the pore homogeneity was poor. The result is illustrated in FIG. 2.

### Example 3

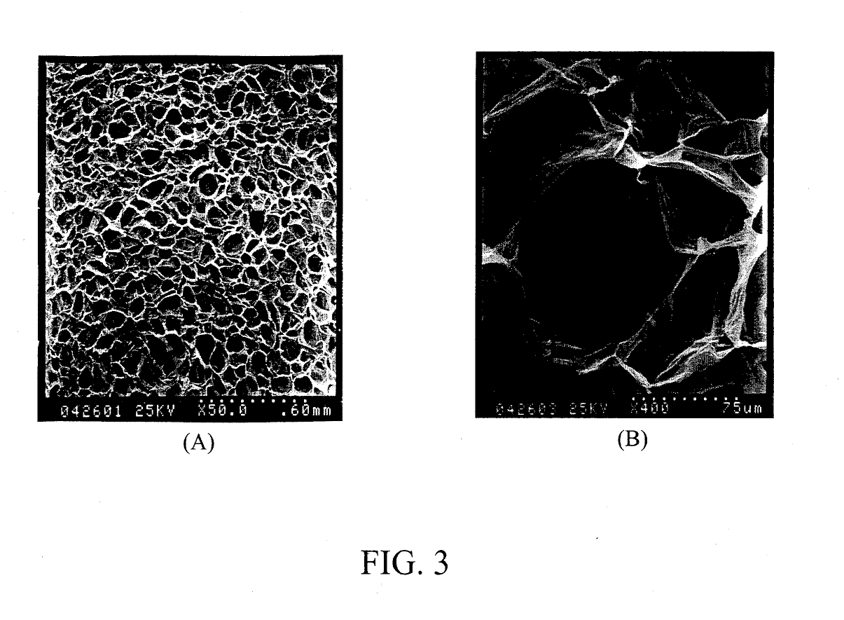

Purified collagen solution was dialyzed against a neutral phosphate buffered saline solution (a 0.02M phosphate buffer containing 0.135M sodium chloride, pH=7.2) to form a neutral collagen solution. The collagen solution was placed in a glass container and incubated in a 37° C. incubator for 24 hours to form a reconstituted collagen gel matrix. The collagen gel matrix was frozen with a rapid temperature reduction rate (the temperature reduction rate is 20° C. per minute) to a temperature of −20° C. and then lyophilized until the matrix was dried. The porous collagen matrix with good pore homogeneity was obtained and the range of the pore size was between 100 and 125 μm. The result is illustrated in FIG. 3.

### Example 4

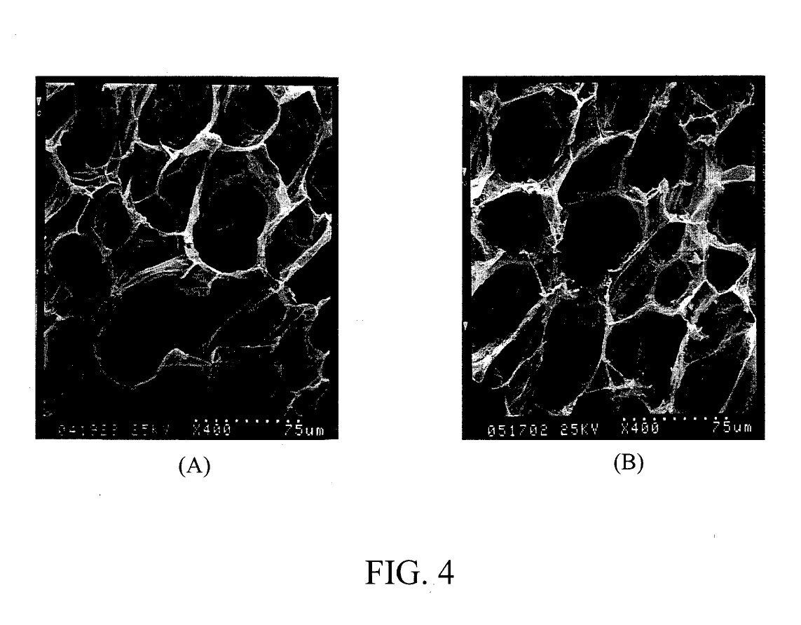

Collagen matrices were prepared by the process described in Example 3 with the modifications to the concentration of sodium chloride added in neutral phosphate buffer to 0.5 and 1.0M. The porous collagen matrices with pore sizes of 30 to 40 μm were obtained. The result is illustrated in FIG. 4.

### Example 5

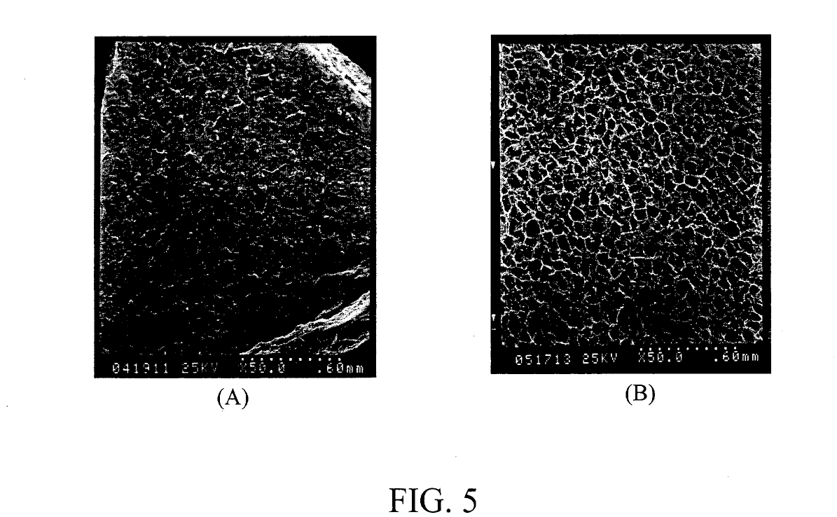

The collagen matrices after lyophilization were immersed in pure water, neutral phosphate buffer, 2% glutaraldehyde, 50% ethanol, 75% ethanol and absolute ethanol, respectively. FIG. 5 shows the influences of the solutions on the pore structure on the surface of the matrices. The ratios of shrinkage along the surface diameter and the thickness and the superficial condensed layer of the matrices were measured. The results are presented in Table 2.

TABLE 2
The ratios of shrinkage and superficial condensed layer of the
porous collagen matrices after the treatment of different solutions

| | Solvent | | | | | |
|---|---|---|---|---|---|---|
| | Absolute | | | 2% | | |
| Ratio (%) | Ethanol | 75% Ethanol | 50% Ethanol | Glutaraldehyde | PBS | Water |
| Shrinkage of | 0.4 ± 0.2 | 3.0 ± 1.9 | 4.5 ± 1.8 | 2.5 ± 1.4 | 20.8 ± 11.2 | 29.9 ± 1.2 |
| diameter (%) | | | | | | |
| Shrinkage of | 1.7 ± 0.1 | 23.0 ± 0.4 | 33.1 ± 1.6 | 17.9 ± 0.2 | 38.5 ± 2.9 | 56.8 ± 5.2 |
| thickness (%) | | | | | | |
| Superficial | 0 | 3.3 | 5.0 | 2.5 | 39.3 | 45.8 |
| condensed | | | | | | |
| layer (%) | | | | | | |

The results of Table 2 demonstrate that the collagen matrices immersed in absolute ethanol exhibit the smallest shrinkage ratio, do not form a compact surface and have better stability of pore structure, which can maintain the original pore size and pore structure of the collagen matrices.

### Example 6

Collagen matrices prepared according to Example 3 were subjected to static contact angle measurements. The Sessile drop method according to Good, R. J. was applied to the contact angle measurement, in which the initial static contact angles were instantly measured as the tested solvents dropped onto the surface of the lyophilized porous collagen matrix. The results are presented in Table 3.

TABLE 3
Contact angle of droplets on the surface of collagen matrix

| | pure | absolute | | | | N,N-dimethyl | dimethyl |
|---|---|---|---|---|---|---|---|
| Droplet | water | ethanol | acetone | acetonitrile | chloroform | formamide | sulfoxide |
| Contact | 78.5 ± 0.8 | n.d.* | n.d.* | n.d.* | n.d.* | n.d.* | n.d.* |
| angle | | | | | | | |

*n.d.: As the droplet of organic solvents indicated touched the surface of collagen matrix, it quickly spread out and the contact angle was not detectable (almost zero).

The results of Table 3 demonstrate that the contact angle of a droplet of certain solvents, such as absolute ethanol, acetone or others on the surface of the collagen matrix, is almost zero, while the water droplet exhibits apparent contact angles. Through series of scientific studies and confirmation, the results indicate that the solvents with static contact angles of near zero can quickly penetrate into the porous collagen matrix once they are in contact with each other to stabilize the porous structure of the collagen matrix. The mechanism of stabilization of the porous structure is due to filling of the porous spaces by the organic solvents initially. The results correlate well with the extent of shrinkage of the porous collagen matrix in Example 5.

### Example 7

The stability and shrinkage of four collagen matrices obtained by the following methods are compared:

(1) Collagen matrix prepared according to Example 3 (“PCM”).

(2) Collagen matrix prepared according to Example 3+Example 5 (immersed in absolute ethanol; “PCM-AE”).

(3) Collagen matrix prepared according to Example 3 and U.S. Pat. No. 4,233,360 (in which formaldehyde was used as a cross-linking agent of the collagen gel matrix; “PCM-F”).

(4) Collagen matrix prepared according to Example 3 and U.S. Pat. No. 5,514,181 (in which alcohol was used in the lyophilization of the collagen matrix as an anti-freeze agent; “E-PCM”).

A. Differential Scanning Calorimetric (DSC) Analysis

The four collagen matrices were subject to DSC analysis, and the results are shown in Table 4.

TABLE 4
DSC analysis of collagen matrices prepared according to the
subject invention and U.S. Pat. Nos. 4,233,360 and 5,514,181.

| Collagen | | | | |
|---|---|---|---|---|
| Matrix | PCM | PCM-AE | PCM-F | E-PCM |
| Degradation | 89.6 ± 1.3 | 98.9 ± 0.5 | 110.3 ± 1.5 | 91.9 ± 0.2 |
| Temperature | | | | |
| (° C.) | | | | |

According to the results of DSC analysis, it was understood that the structural strength of the collagen matrix obtained according to the subject invention and that obtained by use of a chemical cross-linking agent have higher structural strength. On the other hand, the addition of ethanol in the lyophilization of the collagen matrix did not apparently enhance the structural strength of the collagen matrix.

B. Stability

The stability of the four collagen matrices was compared by immersing them in pure water and measuring the ratio of shrinkage by changes of surface area. The results are shown in Table 5.

TABLE 5
Ratio of shrinkage of collagen matrices prepared according
to the subject invention and U.S. Pat. Nos. 4,233,360 and 5,514,181

| Collagen | | | | |
|---|---|---|---|---|
| Matrix | PCM | PCM-AE | PCM-F | E-PCM |
| Ratio of | 34.4 ± 1.6 | 0.2 ± 0.1 | 6.2 ± 5.5 | 42.7 ± 3.0 |
| Shrinkage (%) | | | | |
| Matrix Shape | Bad | Excellent | Not Good | Bad |

According to the results shown in Table 5, it was understood that without the immersion treatment of absolute ethanol or the use of a chemical cross-linking agent, the collagen matrices exhibit apparent shrinkage and the structures thereof have been significantly destroyed. The collagen matrix prepared by use of the chemical cross-linking agent exhibit partial shrinkage and the structure thereof has been partially destroyed. The collagen matrix prepared according to the subject invention does not exhibit shrinkage and the structure thereof is quite intact.

We claim:

1. A process for the preparation of a collagen matrix, comprising providing a neutral or nearly neutral collagen solution, incubating the collagen solution at a temperature of between about 30 and about 45° C. for a period of time sufficient to reconstitute collagen fibrils to obtain a collagen gel matrix, freezing said collagen gel matrix with an appropriate temperature reduction rate to an appropriate freezing temperature, lyophilizing said matrix to form a porous collagen matrix and treating the lyophilized collagen matrix with an organic solvent that can quickly penetrate into the collagen matrix to prevent the shrinkage thereof.

2. The process according to claim 1, wherein the collagen solution is incubated at about 37° C. to form a collagen gel matrix.

3. The process according to claim 1, wherein the organic solvent is selected from the group consisting of an alcohol, a ketone, acetonitrile, chloroform, N,N-dimethylformamide and dimethyl sulfoxide.

4. The process according to claim 3, wherein the alcohol is absolute ethanol.

5. A process for the preparation of a collagen matrix, comprising
(a) providing a neutral or nearly neutral collagen solution;
(b) incubating the collagen solution at a temperature of between about 30 and about 45° C. for a period of time sufficient to reconstitute collagen fibrils to obtain a collagen gel matrix,
(c) freezing said collagen gel matrix with an appropriate temperature reduction rate to an appropriate freezing temperature;
(d) lyophilizing said matrix to form a porous collagen matrix, and
(e) treating the lyophilized collagen matrix with an organic solvent that can quickly penetrate into the collagen matrix to prevent the shrinkage thereof,
wherein at least one of the collagen solution, the freezing temperature and the temperature reduction rate is selected or controlled to obtain a collagen matrix with different pore sizes.

6. The process according to claim 5, wherein the collagen solution is prepared by adjusting the collagen solution to a neutral or nearly neutral collagen solution thereby obtaining a porous collagen matrix with different pore sizes.

7. The process according to claim 6, wherein the collagen solution in step (a) is prepared by dialyzing collagen into a neutral salt buffer to form a neutral or nearly neutral collagen solution to obtain a porous collagen matrix with different pore sizes.

8. The process according to claim 7, wherein the neutral salt buffer is a phosphate buffered saline solution.

9. The process according to claim 5, wherein the organic solvent is selected from the group consisting of an alcohol, a ketone, acetonitrile, chloroform, N,N-dimethylformamide and dimethyl sulfoxide.

10. The process according to claim 9, wherein the alcohol is absolute ethanol.

11. The process according to claim 5, wherein step (a) further comprising incorporating a metal salt into the collagen solution to obtain a porous collagen matrix with different pore sizes.

12. The process according to claim 11, wherein the metal salt is sodium chloride.

13. The process according to claim 5, wherein the collagen solution is incubated at about 37° C. to obtain a collagen gel matrix.

14. The process according to claim 5, wherein the matrix in step (c) is frozen at a final temperature of about −20° C. with a rapid temperature reduction rate more than about −5° C. per minute to obtain a porous collagen matrix with good pore homogeneity.

15. A porous collagen matrix prepared by the process according to claim 1.

16. A porous collagen matrix prepared by the process according to claim 3.

17. A porous collagen matrix prepared by the process according to claim 5.

18. A porous collagen matrix prepared by the process according to claim 8.

19. A porous collagen matrix prepared by the process according to claim 10.

20. A porous collagen matrix prepared by the process according to claim 12.

21. A porous collagen matrix prepared by the process according to claim 13.

22. A porous collagen matrix prepared by the process according to claim 14.
